(19) United States
(12) Patent Application Publication
(10) Pub. No.: US 2026/0334948 A1
(43) Pub. Date: Oct. 29, 2026

(54) HEMOSTASIS CLIP

(71) Applicant: BOSTON SCIENTIFIC SCIMED, INC., Maple Grove, MN (US)

(72) Inventors: Ericka Fabiola ALVAREZ CASTRO, San Jose (CR); Fernando VALERIO GUERRERO, Alajuela (CR); Andrés MÁRQUEZ FERNANDEZ, San José de Alajuela (CR); Gonzalo Jose SAENZ VILLALOBOS, Alajuela (CR); Rafael BERENZON, Cartago (CR); Viviana ARTAVIA SALAS, Heredia (CR); Oscar Andres SEGURA ELIZONDO, San Jose (CR); Jessica BONILLA MORA, Alajuela (CR); Max Alejandro RAMIREZ UGALDE, Alajuela (CR); Marco Vinicio ARIAS ALPIZAR, Heredia (CR); Emma Cristina QUESADA MATAMOROS, Heredia (CR); Gustavo CARVAJAL BONILLA, Heredia (CR); Emanuel CHAVES PIZARRO, Cartago (CR); Valeria MAYORGA CERVANTES, San Jose (CR); Jocelyn de los Angeles VILLALOBOS AREVALO, Alajuela (CR); Mauro VIQUEZ GONZALEZ, Heredia (CR)

(21) Appl. No.: 19/643,464

(22) Filed: Apr. 9, 2026

Related U.S. Application Data
(60) Provisional application No. 63/796,041, filed on Apr. 28, 2025.

(51) Int. Cl. A61B 17/08; A61B 1/018; A61B 17/00

(52) U.S. Cl. CPC A61B 17/083; A61B 1/018; A61B 2017/00367

(57) ABSTRACT

A device includes a capsule; clip arms; and a core member. Proximal ends of the arms are slidably received to move the arms between open and closed configurations. The member is bent into a configuration with a proximal portion receiving an enlarged distal end of a control member and a distal portion including first and second planar surfaces between which locking wings are received. The opening deforms to permit a proximal passage of the enlarged distal end therethrough during a device deployment when subject to a force exceeding a predetermined threshold value. Each of the wings connects to the first and/or second surfaces via a bent portion which deforms during wings'movement from unlocked toward locked configurations. A bent portion length is selected so that a force required to move the wings from the unlocked toward locked configurations is less than the threshold value for deforming the opening.

### PRIORITY CLAIM

The present disclosure claims priority to U.S. Provisional Patent Application Ser. No. 63/796,041 filed Apr. 28, 2025; the disclosure of which is incorporated herewith by reference.

### FIELD

The present disclosure relates to endoscopic devices and, in particular, relates to endoscopic clipping devices for treating tissue along the gastrointestinal tract.

### BACKGROUND

Physicians have become more willing to perform aggressive interventional and therapeutic endoscopic gastrointestinal (GI) procedures, which may increase the risk of perforating the wall of the GI tract or may require closure of the GI tract wall as part of the procedure. Such procedures may include, for example, the removal of large lesions, tunneling under the mucosal layer of the GI tract to treat issues below the mucosa, full thickness removal of tissue, treatment of issues on other organs by passing outside of the GI tract, and endoscopic treatment/repair of post-surgical issues (e.g., post-surgical leaks, breakdown of surgical staple lines, and anastomotic leaks). Currently, tissue openings may be closed via endoscope closure devices such as, for example, hemostasis clips inserted through an endoscope. In some cases, however, current endoscopic closure devices may be difficult to use, time consuming to position, or insufficient for certain perforations, conditions, and anatomies.

### SUMMARY

The present disclosure relates to a device for clipping tissue. The device includes a capsule extending longitudinally from a proximal end to a distal end and including a channel extending therethrough; a pair of clip arms, proximal ends of which are slidably received within the channel of the capsule to move the clip arms between an open configuration, in which distal ends thereof are separated from one another, and a closed configuration, in which the distal ends thereof are drawn toward one another; and a core member connecting the proximal ends of the clip arms and slidably received within the capsule to move the clip arms between the open and closed configurations. The core member is formed of a stamped sheet of metal that is bent into a configuration including a proximal portion defining a cavity configured to receive an enlarged distal end of a control member therewithin and a distal portion including first and second planar surfaces between which a pair of locking wings are received, the proximal portion includes a proximal opening through which a remaining length of the control member is configured to extend, the proximal opening configured to deform to permit a proximal passage of the enlarged distal end therethrough during a deployment of the device when subject to a force exceeding a predetermined threshold value, each of the locking wings connected to at least one of the first and second planar surfaces via a bent portion which deforms during movement of the locking wings from an unlocked configuration toward a locked configuration in which the locking wings are moved radially outward to engage a corresponding locking structure of the capsule, a length of the bent portion selected so that a force required to move the locking wings from the unlocked configuration toward the locked configuration is less than the predetermined threshold value for deforming the proximal opening.

In an embodiment, the proximal end of the capsule includes a pair of tabs crimped inward, toward a centerline of the capsule.

In an embodiment, the proximal portion of the core member includes a first flap and a second flap defining a portion of the cavity, the first and second flaps angled with respect to a longitudinal axis of the core member, a distance between distal ends of the first and second flaps configured so that, when the first and second flaps slide into contact with the tabs of the capsule, the tabs are pushed radially outward.

In an embodiment, the locking structure of the capsule includes a pair of locking windows extending through a wall thereof, each of the locking windows tapering toward a proximal end thereof to ensure that, when the locking wings are received within the locking windows, the first and second flaps are aligned with the tabs of the capsule to push the tabs radially outward.

In an embodiment, the clip arms are connected to the core member via a rivet received within holes extending through the proximal end of the clip arms and through elongate openings extending through the first and second planar surfaces of the distal portion of the core member.

In an embodiment, in the unlocked configuration, a portion of the locking wings overlap a distal portion of the elongate openings.

In an embodiment, the rivet is slidable from a proximal end of the elongate openings toward a distal end to move the locking wings from the unlocked configuration toward the locked configuration.

In an embodiment, interior longitudinal edges of the locking wings extend along one of a curve and an angle relative to a longitudinal axis of the core member selected so that, when the rivet is slid distally therealong, the locking wings are forced toward the locking configuration.

In addition, the present disclosure relates to a system for clipping tissue. The system includes an insertion device including a catheter extending longitudinally from a proximal end to a distal end and a bushing configured to be mounted over the distal end of the catheter; a clip including a capsule releasably coupled to the catheter via the bushing and a pair of clip arms, proximal ends of which are connected to one another via a core member slidably received within a channel of the capsule to move the clip arms between an open configuration, in which distal ends of the clip arms are separated from one another to receive a tissue therebetween, and a closed configuration, in which distal ends of the clip arms are drawn toward one another to grip a tissue therebetween, the core member formed of a stamped sheet of metal that is bent into a configuration including a proximal portion defining a cavity and a distal portion including first and second planar surfaces between which a pair of locking wings are received, each of the locking wings connected to at least one of the first and second planar surfaces via a bent portion configured to deform when the locking wings is moved from an unlocked configuration toward a locked configuration, in which the locking wings are moved radially outward to engage a corresponding locking structure of the capsule; and a control member including an enlarged distal end received within the cavity of the core member and a remaining length passing through a proximal opening of the core member to extend through the bushing and catheter to a proximal end of the insertion device, the proximal opening configured to deform to permit a proximal passage of the enlarged distal end therethrough during a deployment of the clip, when subject to a force exceeding a predetermined threshold value, a length of the bent portion selected so that a force required to move the locking wings from the unlocked configuration toward the locked configuration is less than the predetermined threshold value for deforming the proximal opening.

In an embodiment, the bushing includes a dimple projecting into a channel of the bushing, the dimple sized, shaped, and configured to prevent the distal end of the catheter from moving distally therepast.

In an embodiment, the capsule includes a pair of tabs crimped radially inward configured to engage corresponding engaging feature at a distal end of the bushing.

In an embodiment, the proximal portion of the core member includes a first and second flap configured to engage the tabs of the capsule to move the tabs radially outward.

In an embodiment, the capsule including a pair of locking windows extending through a wall of the capsule and configured to receive the locking wings therein, in the locked configuration, each of the locking windows tapering toward a proximal end thereof so that, when the locking wings engage the locking windows, the core member is accurately aligned with the capsule to ensure that the first and second tabs engage the tabs to move the tabs radially outward.

In an embodiment, the clip further comprises a rivet received within holes extending through proximal ends of the clip arms and through elongated openings extending through the first and second planar surfaces to connect the clip arms to the core member.

In an embodiment, the rivet is movable from a proximal end of the elongated openings toward a distal end of the elongated openings to engage a portion of the locking wings to slide along a portion of the locking wings overlapping a distal portion of the elongated openings to push the locking wings radially outward toward the locking configuration.

Furthermore, the present disclosure relates to a method for treating target tissue. The method includes inserting a clip through a working channel of an endoscope to a target site within a body via a catheter releasably coupled to the clip via a bushing, the clip including a capsule and clip arms, proximal end of the clip arms connected to one another via a core member slidably received within the capsule; moving the clip between an open configuration, in which distal ends of the clip arms are separated from one another, to a closed configuration, in which the distal ends of the clip arms are drawn toward one another, until a target tissue is clipped between the clip arms, as desired, the clip moved between the open and closed configurations by moving a control member coupled to the core member distally and proximally relative to the capsule, an enlarged distal end of the control member received within a cavity of the core member and a remaining length of the control member extending proximally from the core member via a proximal opening of the core member coupled to the core member; drawing the control member proximally relative to the capsule until engaging features along a distal portion of the clip arms abut a distal face of the capsule preventing further proximal movement of the clip arms relative to the capsule; locking the clip arms toward the closed configuration, clipped over the target tissue, by drawing the control member further proximally relative to the capsule until a rivet received within openings at proximal ends of the clip arms and within an elongated opening of the core member is moved from a proximal end of the elongated opening toward a distal end of the elongated opening, sliding along interior longitudinal edges of a pair of locking wings to force the locking wings radially outward toward a locked configuration in which the locking wings extend into locking windows of the capsule; and deploying the clip by drawing the control member further proximally relative to the capsule until a force exerted on a proximal opening of the control member via the enlarged distal exceeds a predetermined threshold value such that the proximal opening deforms to permit a proximal passage of the enlarged distal end of the control member therethrough.

In an embodiment, the bushing is mounted over the distal end of the catheter, the bushing including a dimple projecting into a channel thereof to prevent the distal end of the catheter from moving distally therepast.

In an embodiment, when the locking wings of the core member are received within the locking windows of the capsule, first and second flaps along a proximal portion of the core member engage tabs at a proximal end of the capsule, which are crimped radially inward to engage a corresponding portion of the bushing, to move the tabs radially outward, out of engagement with the corresponding portion of the bushing.

In an embodiment, the locking windows taper toward a proximal end thereof to ensure appropriate alignment of the first and second flaps of the core member with the tabs of the capsule.

In an embodiment, the core member is formed of a stamped sheet of metal that is bent into a configuration including a proximal portion defining the cavity and a distal portion connected to the proximal end of the clip arms, the distal portion including first and second planar surfaces between which the locking wings are received, each of the locking wings connected to at least one of the first and second planar surfaces via a bent portion, a length of the bent portion selected so that a force required to move the locking wings from an unlocked configuration toward the locked configuration is less than the predetermined threshold value required to deform the proximal opening of the core member during clip deployment.

### BRIEF DESCRIPTION OF THE DRAWINGS

FIG. 1 shows a partially transparent longitudinal side view of a distal portion of a clipping system according to an exemplary embodiment of the present disclosure.

FIG. 2 shows an exploded longitudinal side view of the distal portion of the exemplary system of FIG. 1.

FIG. 3 shows another exploded longitudinal side view of the distal portion of the exemplary system of FIG. 2, rotated approximately 90 degrees about a longitudinal axis of the system.

FIG. 4 shows a perspective view of a bushing of an insertion device according to the exemplary system of FIG. 1.

FIG. 5 shows a side view of the bushing of FIG. 4.

FIG. 6 shows a longitudinal cross-sectional view of the bushing of FIG. 4.

FIG. 7 shows a perspective view of a capsule of a clip according to the exemplary system of FIG. 1.

FIG. 8 shows a perspective view of a core member of the clip according to the exemplary system of FIG. 1.

FIG. 9 shows a side view of the core member of FIG. 8.

FIG. 10 shows a plan view of the core member of FIG. 8 from a proximal end thereof.

FIG. 11 shows a side view of the core member of FIG. 8, illustrating a deformation of a proximal opening thereof.

FIG. 12 shows another side view of the core member of FIG. 8.

FIG. 13 shows a cross-sectional view of the core member of FIG. 4, along line A-A.

FIG. 14 shows a side view of a core member according to an alternate embodiment of the system of FIG. 1.

FIG. 15 shows a side view of a core member if Fig. according to another alternate embodiment of the system of FIG. 1.

FIG. 16 shows a partially transparent longitudinal side view of a distal portion of the system of FIG. 1, in an open configuration.

FIG. 17 shows a partially transparent longitudinal side view of the distal portion of the system of FIG. 1, in a closed configuration.

FIG. 18 shows a partially transparent longitudinal side view of the distal portion of the system of FIG. 1, in a locked configuration.

FIG. 19 shows a partially transparent longitudinal side view of the distal portion of the system of FIG. 1, wherein a control wire is separated from the clip during a deployment process.

FIG. 20 shows a partially transparent longitudinal side view of the distal portion of the system of FIG. 1, wherein an insertion device is separated from the clip during the deployment process.

FIG. 21 shows a partially transparent longitudinal side view of a deployed clip according to the exemplary system of FIG. 1.

### DETAILED DESCRIPTION

The present disclosure may be further understood with reference to the following description and the appended drawings, wherein like elements are referred to with the same reference numerals. The present disclosure is directed to an endoscopic clipping system for treating internal tissue perforations, defects, and/or bleeds. Exemplary embodiments of the present disclosure describe a clipping system comprising a clip releasably coupled to a proximal portion of the system, the clip including a pair of clip arms, proximal ends of which are slidable within a capsule to move the clip arms between an open configuration, in which distal ends of the clip arms are separated from one another to receive target tissue therebetween, and a closed configuration, in which the clip arms are drawn into the capsule so that the distal ends of the clip arms are moved toward one another to grip the target tissue therebetween.

The exemplary clipping system further comprises a core member which couples the proximal ends of the clip arms to one another and to a distal end of a control member moved by an operator of the clipping system (e.g., a surgeon) to move the clip between the open and closed configurations and, upon clipping of the clip over a target tissue as desired, toward a deployment of the clip. The exemplary core member includes a locking feature or structure which, when the deployment is initiated, locks the clip arms relative to the capsule in the closed configuration. The exemplary locking feature allows the use of a capsule that is shorter, reducing the overall length of a deployed clip as compared to current hemostasis clips including capsules in which a clip is locked via locking structures on the clip arms themselves. As will be understood by those of skill in the art, shorter deployed clips may improve visualization of the target site and allow for better maneuverability when, for example, placing multiple clips.

Some current clip designs also create shed parts such as, for example, a distal end of a control member which is separated from a length of the control member during deployment of the clip. The shed parts may be left in the body to pass through the GI tract naturally. As the treatment of larger defect cases such as Peroral Endoscopic Myotomy (POEM) and Endoscopic Submucosal Dissection (ESD) become more prevalent, physicians may prefer clips that do not generate shed parts to eliminate the possibility of shed parts causing harm, e.g., by becoming embedded in the defects, etc. A core member according to an exemplary embodiment is configured to eliminate shed parts by, for example, facilitating removal of a control wire in its entirety (i.e., including a distal end thereof), upon deployment of the clip. In an exemplary embodiment, the core member includes a proximal opening that is configured to deform to allow passage of the distal end of the control wire therethrough during deployment of the clip. Thus, the control wire may be removed from the body, upon deployment of the clip, along with the proximal portion of the clipping system. It will be understood by those of skill in the art that the terms proximal and distal as used herein, are intended to refer to a direction toward and away from, respectively, a user of the system.

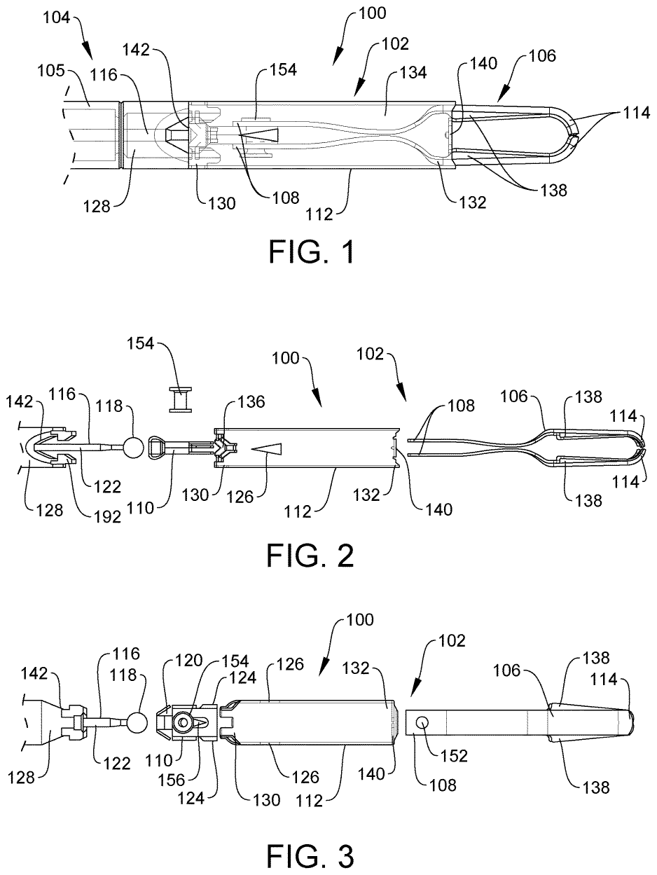

FIGS. 1-21 show a clipping system 100 for treating tissue defects comprising a clip 102 releasably coupled to an insertion device 104 including, for example, a catheter 105. The clip 102 includes a pair of clip arms 106, proximal ends 108 of which are coupled to one another via a core member 110 which is slidably received within a capsule 112 to move the clip 102 between an open configuration, in which distal ends 114 of the clip arms 106 are separated from one another to receive tissue therebetween, and a closed configuration, in which the distal ends 114 of the clip arms 106 are drawn toward one another to grip tissue therebetween. The clip arms 106 are moved between the open and closed configurations via a control member 116 including an enlarged distal end 118 which is received within a cavity 120 of the core member 110 while a remaining length 122 of the control member 116 extends proximally therefrom to pass out of the core member 110, through the insertion device 104, to a proximal end that is accessible to a user of the system 100 for actuation via an actuator (not shown). Thus, a longitudinal movement of the control member 116 relative to the capsule 112 moves the clip 102 between the open and the closed configurations.

As will be described in further detail below, the core member 110 is configured to facilitate both a locking of the clip 102 toward the closed configuration and a separation of the clip 102 from the insertion device 104 (including the control member 116) during a deployment of the clip 102. It will be understood by those of skill in the art that the exemplary core member 110 allows the deployed clip 102 to have a shorter length relative to current hemostasis clips and does not result in any shed parts.

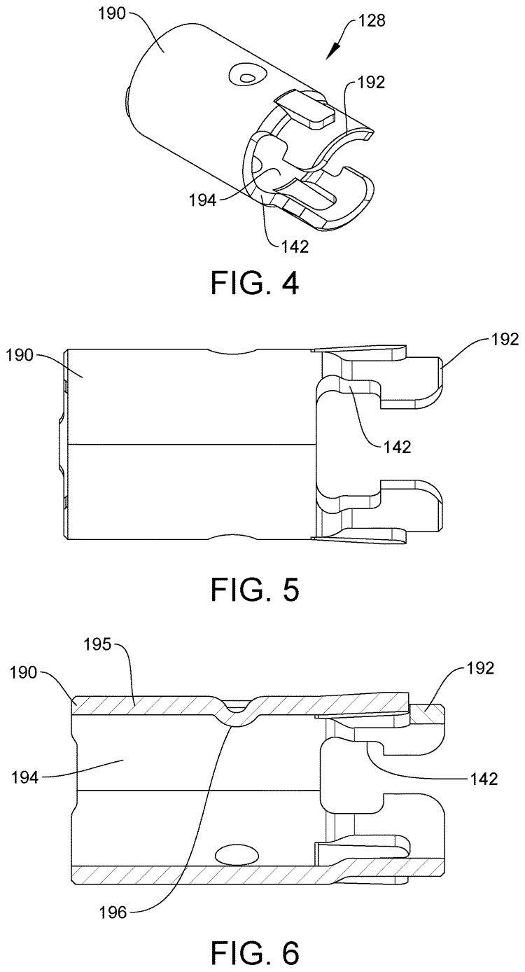

As described above and as shown in FIG. 1, the insertion device 104 may include the catheter 105 for inserting the clip 102 to a target area within a body. According to an exemplary embodiment and as shown in the exploded views of the system 100, the catheter 105 may be releasably coupled to the clip 102 via a bushing 128 that is configured to be mounted over and/or affixed to a distal end of the catheter 105. As shown in FIGS. 4-6, the bushing 128 extends longitudinally from a proximal end 190 configured to be mounted over the distal end of the catheter 105 to a distal end 192 configured to releasably engage the capsule 112 of the clip 102. The bushing 128 includes a channel 194 extending longitudinally therethrough so that the remaining length 122 of the control member 116 may extend through the channel 194 of the bushing 128 and through the catheter 105 to the proximal end of the insertion device 104.

In an exemplary embodiment, the bushing 128 may include a dimple 196 (see FIG. 6) or other structure which projects into the channel 194 to reduce a cross-sectional area thereof at a selected location. The dimple 196 may be formed, for example, by pushing a portion of a wall 195 of the bushing 128 radially inward-e.g., toward a centerline of the bushing 128—during, for example, a stamping of the bushing 128. The dimple 196 or other inward projection may be configured such that, when the bushing 128 is mounted over the distal end of the catheter 105, the catheter 105 contacts the dimple 196 and is prevented from being moved distally past the dimple 196. In other words, the dimple 196 acts as a stop preventing any distal movement of the catheter 105 therepast.

In an exemplary embodiment, the bushing 128 may include a pair of dimples 196 diametrically opposing one another or any other desired arrangement of inwardly projecting structures to prevent the catheter 105 from moving distally past a desired distal-most position of the catheter 105 relative to the bushing 128. It will be understood by those of skill in the art, however, that while the dimple(s) 196 keep the catheter 105 (or other elongate member of the insertion device 104) from being moved distally therepast, the dimple(s) 196 do not interfere with longitudinal movement of the control member 116 relative to the bushing 128. It will also be understood by those of skill in the art that although the exemplary embodiments show and describe the dimple 196, the bushing 128 may include any of a variety of structures extending into the channel 194 to prevent distal movement of the distal end of the catheter 105 therepast.

The distal end 192 of the bushing 128 may include an engaging portion 142 configured to releasably engage a corresponding portion of the capsule 112, as will be described in further detail below. The engaging portion 142 may be configured as, for example, a groove, recess, and/or cut-out sized and shaped to receive a portion of the capsule 112 therein, as will be described in further detail below. In an exemplary embodiment, the bushing 128 may include a pair of engaging portions 142 diametrically opposing one another. The bushing 128 may be releasably coupled to the capsule 112 of the clip 102 via the engaging portions 142 and via a friction fit therebetween.

It will be understood by those of skill in the art, however, that although the exemplary embodiment describes the insertion device 104 as including the catheter 105, the insertion device 104 may include any flexible elongate member that is insertable through, for example, tortuous paths of a body lumen to reach a target site. For example, it may be desired that the insertion device is sufficiently flexible to be inserted to a target site within the body through a working channel of a flexible endoscope. The bushing 128 may be similarly affixed thereto to releasably couple the insertion device 104 to the clip 102.

As shown in FIGS. 2-3, the control member 116 includes an enlarged distal end 118 with a remaining length 122 extending proximally therefrom to couple to, for example, an actuator on a handle of the device. The enlarged distal end 118 is configured to be received within a portion of the core member 110 so that the enlarged distal end 118 is connected to the clip arms 106, as will be described in further detail below. The remaining length 122 extends proximally therefrom, through the bushing 128 and the catheter 105, to the proximal end of the insertion device 104. It will be understood by those of skill in the art that the enlarged distal end 118 has a width in at least one dimension larger than that of a proximal opening 180 of the core member 110 through which the control member 116 passes through to extend to the proximal end of the insertion device 104. This prevents the enlarged distal end 118 of this embodiment from passing proximally out of the proximal opening 180 in the core member 110 as the clip 102 is moved between the open and the closed configurations. In an exemplary embodiment, the enlarged distal end 118 may be ball-shaped.

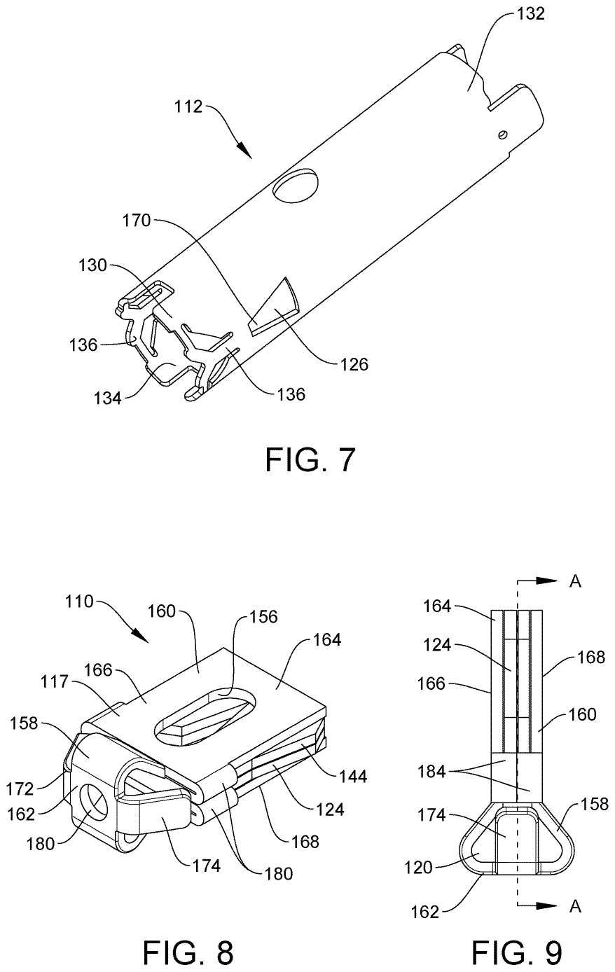

The capsule 112 of the clip 102 extends from a proximal end 130 to a distal end 132 and includes a channel 134 extending therethrough. In one embodiment, the proximal end 130 of the capsule 112 is releasably coupled to the bushing 128 via, for example, one or more tabs 136 each of which is crimped radially inward to engage the engaging portion 142 of the bushing 128. In one embodiment, the capsule 112 includes a pair of tabs 136 diametrically opposing one another, where each of the tabs 136 is configured to engage a corresponding one of the engaging portions 142 of the bushing 128. The capsule 112 also includes locking windows 126 extending laterally through a capsule wall to engage locking features 124 of the core member 110. In an exemplary embodiment, as shown in FIG. 7, the locking windows 126 have a substantially triangular shape which tapers toward a proximal end 170. As will be described in further detail below, the tapering shape of the locking windows 126 facilitates accurate alignment of the core member 110 relative to the capsule 112 during deployment of the clip 102.

Each of the clip arms 106 extends from the proximal end 108 to the distal end 114. As described above, the proximal ends 108 are slidably received within the channel 134 so that the clip arms 106 may be moved relative to the capsule 112 between the open and the closed configurations via manipulation of the control member 116. In one embodiment, the clip arms 106 are biased toward the open configuration so that, when advanced distally out of the capsule 112, the clip arms 106 move apart from one another into the open configuration under their natural bias. When the clip arms 106 are drawn proximally into the capsule 112, the clip arms 106 are constrained by the wall of the capsule 112 and drawn together toward the closed configuration, with the distal ends 114 adjacent to one another. Those skilled in the art will understand that a number of other mechanisms for opening and closing the clip arms 106 may be employed.

Each of the clip arms 106 of this embodiment also includes engaging features 138 extending therefrom configured to engage a portion of the capsule 112 so that when the engaging features 138 engage the capsule 112, the clip arms 106 are prevented from being moved further proximally into the capsule 112. In one embodiment, the engaging features 138 extend laterally outward from distal portions of the clip arms 106, so that the distal portions of the clip arms 106 have a width greater than proximal portions of the clip arms 106 and greater than a diameter of a distal opening of the capsule 112. Thus, the proximal portions of the clip arms 106 are sized to permit themselves to be drawn proximally into the capsule 112 while the portions of the clip arms 106 extending distally from the engaging features 138, are too wide to be drawn into the capsule 112.

Thus, as the clip arms 106 are drawn proximally into the capsule 112, the engaging features 138 abut a portion of a distal face 140 of the capsule 112 preventing further proximal movement of the clip arms 106 relative to the capsule 112. The engaging features 138 are positioned along the clip arms 106 so that, at the point where the engaging features 138 have engaged the capsule 112, the clip arms 106 have been drawn sufficiently proximally into the capsule 112 to draw the clip arms 106 together, into the closed configuration. In one example, the engaging features 138 are configured as wings extending laterally from longitudinal edges of the clip arms 106.

Each of the proximal ends 108 of the clip arms 106, in this embodiment, includes a hole 152 extending therethrough. The hole 152 is configured to facilitate a coupling of the proximal ends 108 of the clip arms 106 to the core member 110. According to an exemplary embodiment, when the clip arms 106 are diametrically opposed relative to one another, the holes 152 of the clip arms 106 are aligned with one another so that a connector such as, for example, a rivet 154 may be passed through the holes 152 at the proximal ends 108 of the clip arms 106 and through a corresponding elongated opening 156 of the core member 110 to couple the clip arms 106 to the core member 110.

As described above, the core member 110 is sized and configured to be longitudinally slidable within the channel 134 of the capsule 112. In an exemplary embodiment, as shown in FIGS. 8-13, the core member 110 is a one-piece mechanism configured to connect the control member 116 to the clip arms 106 and, according to an exemplary embodiment, is formed of a stamped sheet of material 117 (e.g., metal) sized and shaped so that, when bent and/or folded, as will described in further detail below, the material 117 includes a proximal portion 158 defining the cavity 120 (see FIG. 9), which is configured to receive the enlarged distal end 118 of the control member 116, and a distal portion 160, which defines a locking feature 124 configured to lock the clip arms 106 relative to the capsule 112 in the closed configuration.

The core member 110, in the bent configuration, extends from a proximal end 162 to a distal end 164. According to an exemplary embodiment, the material 117 is bent over the proximal end 162 to include a first surface 166 and a second surface 168. In an exemplary embodiment, the core member 110 may have a length ranging between 0.10 and 0.15 inches and, in one example, may have a length of approximately 0.111 inches. As described above, the core member 110 facilitates both a locking of the clip 102 toward the closed configuration and a deployment of the clip 102 (e.g., separation of the clip 102 from the insertion device 104) so that the clip 102 may have a shorter length relative to current conventional clips.

A portion of the first and second surfaces 166, 168 extending along the proximal portion 158 defines the cavity 120 therebetween. A portion of the first and second surfaces 166, 168 extending along the distal portion 160 of the core member 110 is, in an exemplary embodiment, substantially planar so that the first and second surfaces 166, 168, respectively, extend parallel to one another along a longitudinal axis of the core member 110, which is in alignment with a longitudinal axis of the capsule 112 within which the core member 110 is received in an operative configuration.

Along the distal portion 160, each of the first and second surfaces 166, 168 includes an elongated opening 156 extending therethrough along a central axis. Each elongated opening 156 is elongated along a longitudinal axis of the core member 110 and the elongated openings 156 are aligned with one another along a central axis that extends substantially perpendicular to the first and second surfaces 166, 168 so that the rivet 154 may be passed through the core member 110 via the elongated openings 156. As will be described in further detail below, the elongated opening 156 of each of the first and second surfaces 166, 168 is configured to receive the rivet 154 or other connector which connects the clip arms 106 to the core member 110. The elongated opening 156 is sized, shaped, and configured to permit sliding of the rivet 154 along a length thereof.

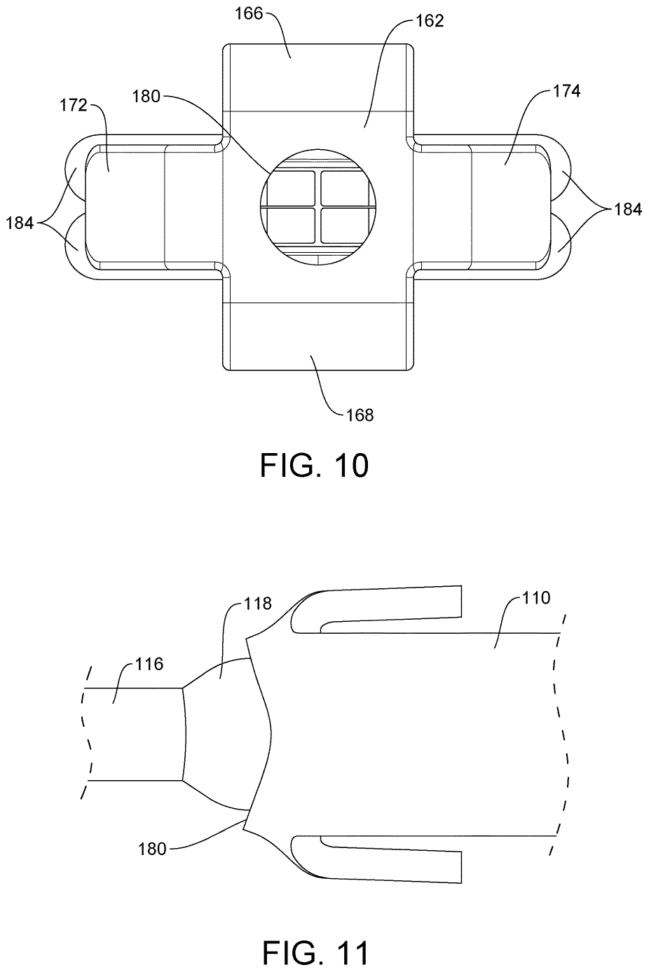

In addition to the first and second surfaces 166, 168, the core member 110 also includes a first flap 172 and a second flap 174 extending from the proximal end 162 and bent relative to the longitudinal axis of the core member 110 toward the distal end 164 to further define the cavity 120. In particular, each of the first and second flaps 172, 174 extends along the proximal portion 158 of the core member 110 so that the cavity 120 is defined between the first and second surfaces 166, 168 and the first and second flaps 172, 174. The proximal end 162 includes a proximal opening 180 extending therethrough along a central axis substantially aligned with the longitudinal axis of the core member 110. The proximal opening 180 is sized and shaped so that, when the enlarged distal end 118 is received within the cavity 120, the control member 116 extends through the proximal opening 180 so that the remaining length 122 of the control member 116 extends proximally to a handle of the device. It will be understood by those of skill in the art that the defined cavity 120 is sized, shaped, and configured to house the enlarged distal end 118 of the control member 116 therein while the proximal opening 180, as shown in FIG. 10, is sized and shaped to prevent the passage of the enlarged distal end 118 therethrough during movement of the clip 102 between the open and the closed configurations.

The proximal opening 180 is configured so that, once the clip 102 has been clipped over the target tissue as desired, the increasing tension applied to the control member 116 will pull the enlarged distal end 118 against a distal side of the proximal opening 180 with increasing force until the material around the perimeter of the proximal opening 180 deforms to permit passage of the enlarged distal end 118 of the control member 116 therethrough, as shown in FIG. 11. According to an exemplary embodiment, the proximal opening 180 is configured to deform when subject to a force exceeding a predetermined threshold value.

In particular, the proximal opening 180 is deformed to permit the proximal passage of the enlarged distal end 118 therethrough during deployment of the clip 102—e.g., separation of the clip 102 from the control member 116. According to an exemplary embodiment, a thickness of the portion of the material 117 surrounding the proximal opening 180 may be selected to facilitate deformation thereof (i.e., to set the force required to permit the enlarged distal end 118 to be drawn proximally through the proximal opening 180 during the deployment process.

The control member 116 is drawn proximally relative to the core member 110 so that the enlarged distal end 118 continues to apply a force exceeding the predetermined threshold force on the proximal opening 180 and, in particular, along a portion of the material 117 surrounding the proximal opening 180, until the proximal opening 180 is sufficiently deformed to permit passage of the entire enlarged distal end 118 therethrough. Thus, once the enlarged distal end 118 passes through the deformed proximal opening 180, the insertion device and the control member 116 may be separated from the clip 102 and removed from the body, leaving the deployed clip 102 clipped over the target tissue in the body. This reduces the likelihood that broken and/or separated pieces will be generated during deployment. Thus, it will be understood by those of skill in the art that this embodiment minimizes shed parts and the risks associated therewith.

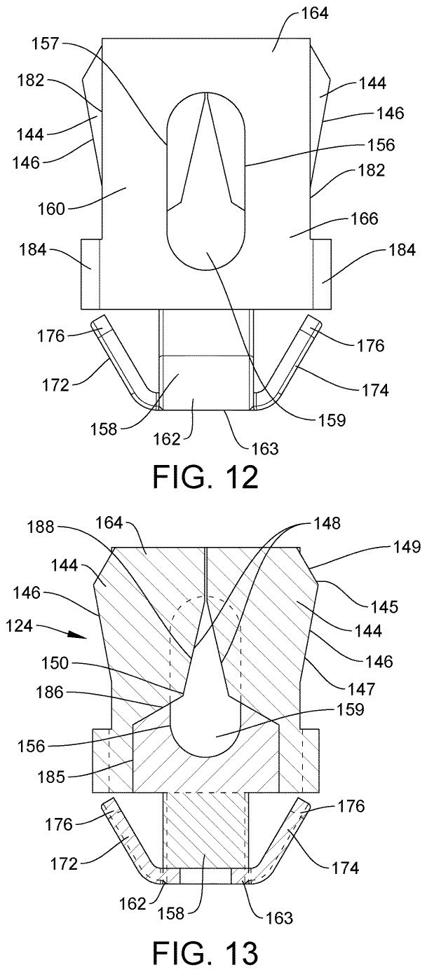

As shown in FIGS. 12-13, the first and second flaps 172, 174 are angled with respect to the longitudinal axis of the core member 110 so that a width of the core member 110 at distal ends 176 (e.g., a distance between the distal ends 176) of the first and second flaps 172, 174 is at least as great as, or greater than, a width of the core member 110 at the proximal end 162 (e.g., a distance between proximal ends of the first and second flaps 172, 174). The first and second flaps 172, 174 are angled so that, when the core member 110 is drawn proximally relative to the capsule 112 during a deployment of the clip 102, the first and second flaps 172, 174 contact and engage the tabs 136, which are crimped radially inwardly relative to the longitudinal capsule to 112 couple the capsule 112 to the bushing 128.

As the angled surfaces of the first and second flaps 172, 174 slide proximally along the tabs 136, engagement between the first and second flaps 172, 174 and the radially inwardly crimped tabs 136 initiate movement of the tabs 136 in a radially outward direction relative to the longitudinal axis of the capsule 112. As will be described in further detail below, as the core member 110 continues to move proximally with respect to the capsule 112, the bent portions 184 will engage the crimped tabs 136 to complete the radially outward movement of the tabs 136, disengaging the tabs 136 from the corresponding engaging portion of the bushing 128.

In an exemplary embodiment, in an unfolded configuration in which the first and second flaps 172, 174 extend substantially perpendicular to a longitudinal axis of the core member 110 in alignment with a proximal face 163 of the core member 110, each of the first and second flaps 172, 174 extends along a length (i.e., distance from the proximal face 163 to the distal end 176 parallel to a longitudinal axis of the clip) of, for example, approximately 0.030 inches. The first and second flaps 172, 174 may be bent inward, from the unfolded configuration toward the distal end 164 of the core member 110 at an angle of approximately 60 degrees. As described above, the first and second flaps 172, 174 should be angled and dimensioned so that the distance between the distal ends 176 facilitates a movement of the inwardly crimped tabs 136 of the capsule 112 toward a radially outward position when engaged therewith.

Portions of the material 117 extending from exterior longitudinal edges 182 of one of the first surface 166 and the second surface 168 may also be bent inward to extend between the first and second surfaces 166, 168 to define the locking feature 124. In an exemplary embodiment, the locking feature 124 is configured as a pair of wings 144 extending between the first and second surfaces 166, 168 and movable between an unlocked configuration, in which the core member 110 is slidable within the capsule 112, and a locked configuration, in which a portion of the wings 144 extend into corresponding locking windows 126 of the capsule 112 to lock the clip arms 106 in the closed configuration.

Each of the pair of wings 144 may be defined via an exterior longitudinal edge 146 and an interior longitudinal edge 148. The interior longitudinal edges 148 of the pair of wings 144 face one another while the exterior longitudinal edges 146 of the pair of wings 144 face away from one another, toward a surface of the channel 134 of the capsule 112. According to an exemplary embodiment, each of the wings 144 is connected to at least one of the first and second surfaces 166, 168 via a bent portion 184 of the material 117 extending along a proximal length of the exterior longitudinal edges 182, 146 of the at least one of the first and second surfaces 166, 168 and the wings 144.

The exterior longitudinal edges 146 may be sized, shaped, and configured to be received within the locking windows 126 of the capsule 112. In an exemplary embodiment, a distal portion of each of the exterior longitudinal edges 146 may include a first portion 147 and a second portion 149 angled with respect to one another and meeting at a point 145 which defines a portion of each of the wings 144 having a greatest width—e.g., largest distance between the exterior longitudinal edge 146 and the interior longitudinal edge 148. The first and second portions 147, 149 of the exterior longitudinal edges 146 may be angled with respect to one another so as to reduce a friction between the capsule 112 and the core member 110, as the core member 110 is moved longitudinally with respect to the capsule 112.

In the unlocked configuration, the points 145 along the exterior longitudinal edges 146 of the pair of wings 144 are separated from one another by a distance smaller than, for example, a diameter of the channel 134 of the capsule 112 so that the core member 110 is free to slide longitudinally within the channel 134 of the capsule 112 to move the clip arms 106 between the open and closed configurations. In the locked configuration, the pair of wings 144 are moved radially outward so that the distance between the exterior longitudinal edges 146 of the wings is greater than the diameter of the channel 134. Thus, in the locked configuration, the wings 144 extend into and engage the locking windows 126 of the capsule 112 locking the clip in the closed configuration.

It will be understood by those of skill in the art that the bent portion 184 connecting the wings 144 to at least one of the first and second surfaces 166, 168 are plastically deformed as the core member 110 is moved from the unlocked configuration toward the locked configuration such that a distal portion of each of the wings 144 is moved radially outward. As will be described in further detail below, during a deployment of the clip 102, the core member 110 is moved proximally with respect to the clip arms 106 so that the rivet 154 slides distally along the interior longitudinal edges 148 of the wings to push the wings 144 radially outward toward the locked configuration.

According to an exemplary embodiment, each of the interior longitudinal edges 148 includes a first portion 185 extending distally from a proximal end of the wing 144, a second portion 186 extending distally from the first portion 185, and a third portion 188 extending distally from a distal end of the second portion 186 to a distal end of the wing 144. Each of the first, second and third portions 185, 186, 188 may be angled with respect to one another to define the interior longitudinal edge 148 such that a width (e.g., a distance between the exterior longitudinal edge 146 and the interior longitudinal edge 148) of each of the wings 144 substantially increases from the proximal end of the wing 144 toward the distal end 164 of the core member 110.

In an exemplary embodiment, the first portion 185 of the interior longitudinal edge 148 extends substantially parallel to the longitudinal axis of the core member 110 so as not to overlap with any portion of the elongated opening 156. The second portion 186 of the interior longitudinal edge 148 is angled with respect to the first portion 185 so that a distal end of the second portion 186 overlaps the elongated opening 156 to define a portion of a space within the elongated openings 156 through which the rivet 154 extends in the unlocked configuration. In particular, the second portion 186 defines a proximal portion 159 of the elongated openings 156 within which the rivet 154 is received in an initial configuration, before deployment, in the unlocked configuration.

In an exemplary embodiment, the first and second portions 185, 186 extend at an angle of 120 degrees relative to one another. It will be understood by those of skill in the art that the angle between the first and second portions 185, 186 may be selected to control a desired level of force required to move the wings 144 from the unlocked to the locked configuration so that this force is at a desired level as compared to the force required to pull the enlarged distal end 118 of the control member 116 out of the proximal opening 180 as described above.

The second portion 186 and the third portion 188 of the interior longitudinal edge 148 of each of the wings 144 are angled with respect to one another such that the third portion 188 overlaps a distal portion 157 of the elongate openings 156. As the user draws the control member 116 proximally relative to the capsule 112, the core member 110 is drawn proximally relative to the rivet 154 so that the rivet 154 is moved from the proximal portion 159 of the elongated opening 156 toward the distal portion 157 to move the core member 110 toward the locked configuration.

As the core member 110 is moved proximally relative to the rivet 154, the rivet 154 is pressed distally against a point 150 along interior longitudinal edges 148 at which the second and third portions 186, 188 of the interior longitudinal edges 148 meet. As the rivet 154 moves past the point 150, the rivet 154 slides distally along and between the angled third portions 188 of the interior longitudinal edges 148, forcing the wings 144 radially outward away from one another, and away from the longitudinal axis of the core member 110 toward the locking configuration. At the same time, the core member 110 is drawn proximally within the capsule 112 so that the wings 144 are drawn into alignment with and project radially out of the locking windows 126 locking the clip in the closed configuration.

In an exemplary embodiment, the bent portions 184 may be sized, shaped, and configured to move the tabs 136 radially outward, during a deployment of the clip 102, to disengage the capsule 112 from the bushing 128. As described above, while the first and second flaps 172, 174 may initiate the radially outward movement of the tabs 136 as the first and second flaps 172, 174 come into contact therewith, the bent portions 184 engage the tabs 136 to complete the radially outward movement thereof. Thus, in an exemplary embodiment, a distance between the bent portions 184 along opposing exterior longitudinal edges 182, 146 of the at least one of the first and second surfaces 166, 168 is selected to facilitate engagement with the tabs 136 as the core member 110 is moved longitudinally relative to the capsule 112 during the deployment of the clip 102.

In one example, this distance may be approximately 0.075 inches. In an exemplary embodiment, a width of the bent portions 184 defined via the bent portions 184 extending between exterior longitudinal edges 182, 146 of the first and second surfaces 166, 168, respectively, should not exceed a width of each of the tabs 136. It will be understood by those of skill in the art, however, that the bent portions 184 may have any of a variety of dimensions so long as the bent portions 184 facilitate the longitudinal movement of the core member 110 within the capsule 112 and the radially outward movement of the tabs 136, as described herein.

A length of the bent portions 184 may also be selected so as to reduce and/or adjust a friction between the bent portions 184 and the channel 134 of the capsule 112. The length of the bent portions 184 may affect the force required to move the core member 110 longitudinally relative to the capsule 112 and, in particular, may affect a force required to move the core member 110 proximally relative to the rivet 154 and the clip arms 106, during a deployment of the clip 102. In an exemplary embodiment, the length of the bent portions 184 may range from between 0.015 inches to 0.040 inches. It will be understood by those of skill in the art, however, that the bent portions 184 may have any of a variety of dimensions so long as the core member 110 is movable relative to the capsule 112 and/or rivet 154 as described above to move the wings 144 from the unlocked configuration toward the locked configuration.

According to an exemplary embodiment, the length of the bent portions 184 extending along and between the wings 144 and one of the first and second surfaces 166, 168 from which the wings 144 extend may be adjusted to adjust an amount of friction between the core member 110 and the capsule 112 and, consequently, a force required to move the core member 110 through the capsule 112. The lengths of the bent portions 184 are, therefore, adjusted to ensure that the force required to move the core member 110 through the capsule 112 as well as the force required to deform the wings 144 and push them radially outward into the locking configuration is lower than that required to pull the enlarged distal end 118 of the control member 116 out of the proximal opening 180 so that the control member 116 will not be separated from the clip 102 before the clip arms have been locked closed over the clipped tissue. As would be understood by those of skill in the art, these dimensions are adjusted to ensure that a force required to lock the clip 102 in the locked configuration is less than the force required to separate the clip 102 from the insertion device 104.

A force required to move the core member 110 from the unlocked configuration toward the locked configuration may be adjusted in any of a number of ways. In an exemplary embodiment, the force required to lock the clip 102 may be adjusted by adjusting a magnitude of the angle at which the first, second and/or third portions 185, 186, 188 of the interior longitudinal edges 148 of the wings 144 meet and/or by adjusting a length of the bent portions 184. It will be understood by those of skill in the art, however, that while the first, second and third portions 185, 186, 188 are described and shown as being angled with respect to one another, portions of the interior longitudinal edges 148 may similarly extend along a curve.

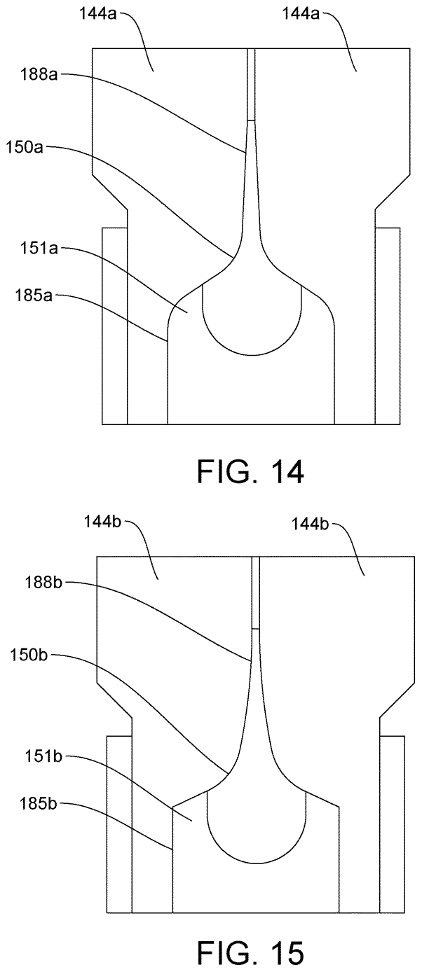

In one example, as shown in FIG. 14, points 150a, 151a at which first and second portions 185a, 186a and second and third portions 186a, 188b, respectively, of an interior longitudinal edge 148a of the wing 144a meet includes a smooth curve, decreasing a force required to deploy the clip 102. In another example, as shown in FIG. 15, a point at which second and third portions 186b, 188b of an interior longitudinal edge 148b meet may be curved while a point 151b at which first portion 185b and second portions 186b meet may be angled. It will be understood by those of skill in the art that the interior and/or exterior longitudinal edges 148, 146 of the wings 144 may be comprised of straight and/or curved portions.

The clip 102 is assembled so that the core member 110 is received between the proximal ends 108 of the clip arms 106, the proximal portion 159 of the elongated openings 156 of the core member 110 aligned with the holes 152 extending through the proximal ends 108 of the clip arms 106. The rivet 154 extends through the holes 152 of the clip arms 106 and through the proximal portions 159 of the elongated openings 156. The core member 110 assembled with the clip arms 106 slidably received within the channel 134 of the capsule 112. As described above, the enlarged distal end 118 of the control member 116 is housed within the cavity 120 within the proximal portion 158 of the core member 110 with the remaining length 122 extending proximally therefrom through the proximal opening 180 at the proximal end 162 of the core member 110 and through the bushing 128 and the catheter 105 to a proximal end accessible via the user or operator of the system 100.

As described above, the clip 102 may be moved between the open and the closed configurations by moving the control member 116 longitudinally relative to the capsule 112. In particular, the control member 116 may be moved distally relative to the capsule 112 to move the clip arms 106 distally out of the capsule 112 toward the open configuration and proximally relative to the capsule 112 to draw the clip arms 106 proximally into the capsule 112 to move the clip 102 toward the closed configuration. Once the clip 102 has been clipped over the target tissue as desired, deployment of the clip 102 may be initiated. Locking of the clip 102 facilitates deployment of the clip 102. In an exemplary embodiment, the control member 116 is drawn proximally until the engaging features 138 at the distal portion of the clip arms 106 abut the distal face 140 of the capsule 112, preventing further proximal movement of the clip arms 106 into the capsule 112.

At this point, increasing proximal tension applied to the control member 116, draws the core member 110 proximally with respect to the clip arms 106 and the capsule 112 so that, as described above, the rivet 154 is moved from the proximal portion 159 of the elongated openings 156 toward the distal portion 157. As the rivet 154 is moved distally along the elongated openings 156, the rivet 154 slides along the interior longitudinal edges 148 of the pair of wings 144, forcing the pair of wings 144 radially outward and into engagement with the locking windows 126.

It will be understood by those of skill in the art that the dimensions of the core member 110 provided above are exemplary and that the core member 110 (e.g., wings, flaps, etc.) may have any of a variety of dimensions so long as the core member 110 is configured to function to lock and/or deploy the clip 102 in the manner described above.

As described above, the locking windows 126 may have a triangular shape so that as the wings 144 extend outward into the locking windows 126, the wings 144 engage the tapering at the proximal end 170 thereof to ensure that the core member 110 is in a desired rotational alignment with the capsule 112. For example, in an exemplary embodiment, the first portion 147 of the exterior longitudinal edge 146 of each of the wings 144 may engage the tapering at the proximal end 170. This desired rotational alignment of the core member 110 within the capsule 112 ensures that, as the clip 102 is moved toward the locked configuration (i.e., the core member 110 is locked relative to the capsule 112), the first and second flaps 172, 174 of the core member 110 are aligned with and come into contact with the tabs 136 of the capsule 112, moving the radially inwardly tabs 136 of the capsule 112 radially outward relative to the longitudinal axis of the capsule 112. As the tabs 136 are moved radially outward, the tabs 136 disengage from the corresponding engaging portion 142 of the bushing 128 so that the insertion device 104 is separated from the clip 102.

Continued application of proximal force to the control member 116 after this point increases tension on the control member 116 and, consequently, increases a force applied by the enlarged distal end 118 against the material 117 surrounding the proximal opening 180 of the core member 110. When this force exerted on the proximal opening 180 by the enlarged distal end 118 exceeds a predetermined threshold force, the proximal opening 180 is deformed to permit passage of the enlarged distal end 118 therethrough. Thus, the insertion device 104 and the control member 116, in its entirety, may be removed from the body, deploying the clip 102 without generating shed parts.

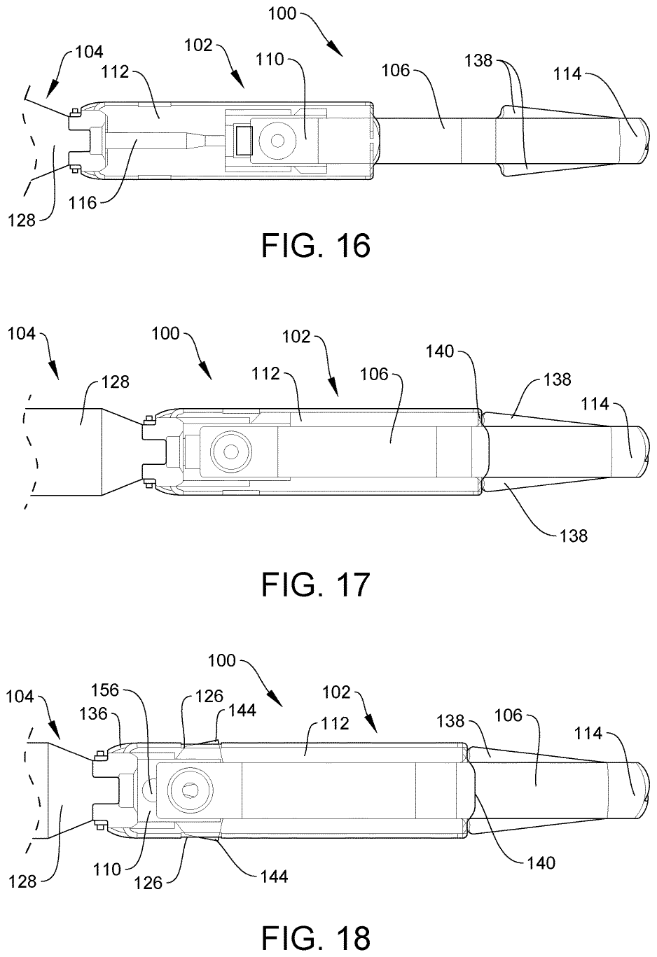

According to an exemplary method utilizing the system 100, as shown in FIGS. 16-21, the clip 102 is inserted through, for example, a working channel of an endoscope to a target site within a body. The clip 102 is inserted to the target site, in the closed configuration, via an insertion device 104 including, for example, a catheter to which the clip 102 is releasably coupled via the bushing 128. Once the clip 102 has reached the target site, the user advances the control member 116 distally relative to the catheter 105 to move the clip arms 106 distally relative to the capsule 112 toward the open configuration, as shown in FIG. 16.

As the clip arms 106 are extended distally from the capsule 112, the clip arms 106 move toward the open configuration, under their natural bias (as they are freed from the constraints of the capsule 112) so that tissue may be received between the distal ends 114 thereof. The user may then draw the control member 116 proximally (or advance the catheter 105 distally over the control member 116) so that, as the clip arms 106 are drawn into the capsule 112, the clip arms 106 are drawn toward one another, toward the closed configuration, to grip the tissue received between the distal ends 114 of the clip arms 106. The clip 102 may be moved between the open and closed configurations, as desired, until a target portion of the tissue is positioned between the clip arms 106 as desired.

When the user is satisfied that the clip 102 is in a desired position gripping the target tissue, the user may initiate deployment of the clip 102 by first locking the clip 102 in the closed configuration and then releasing the clip 102 from the insertion device 104. According to an exemplary embodiment, the user draws the control member 116 proximally so that the engaging features 138 of the clip arms 106 engage the distal face 140 of the capsule 112, as shown in FIG. 17, preventing a further proximal motion of the clip arms 106 relative to the capsule 112. After this point, continued proximal motion of the control member 116, draws the core member 110 proximally relative to the clip arms 106 so that the core member 110 slides proximally relative to the rivet 154. As the core member 110 is moved proximally relative to the clip arms 106, the rivet 154 moves from its position in the proximal portion 159 of the elongated opening 156 of the core member 110 toward the distal portion 157 of the elongated openings 156.

As the rivet 154 slides toward the distal portion of the elongated openings 156, the rivet 154 engages portions of the wings 144 overlapping the elongated openings 156. The rivet 154 slides distally along the angled interior longitudinal edges 148 of the wings 144 pushing the wings 144 from the unlocked configuration radially outward to the locked configuration, as shown in FIG. 18. In the locked configuration, the wings 144 are plastically deformed so that they remain in their radially outward position relative to the longitudinal axis of the core member 110 extending through the locking windows 126 (e.g., the wings 144 extend into and/or through the locking windows 126) and preventing movement of the core member 110 and the clip arms relative to the capsule 112 (i.e., locking the clip in the closed configuration with the distal ends of the clip arms drawn together to grip target tissue). A size and shape of the locking windows 126 ensures that, when the wings 144 of the locking feature 124 of the core member 110 are in the locked configuration, the core member 110 is accurately aligned with the capsule 112 so that the first and second flaps 172, 174 engage the radially inwardly crimped tabs 136 of the capsule 112, forcing the tabs 136 radially outward, out of engagement with the bushing 128.

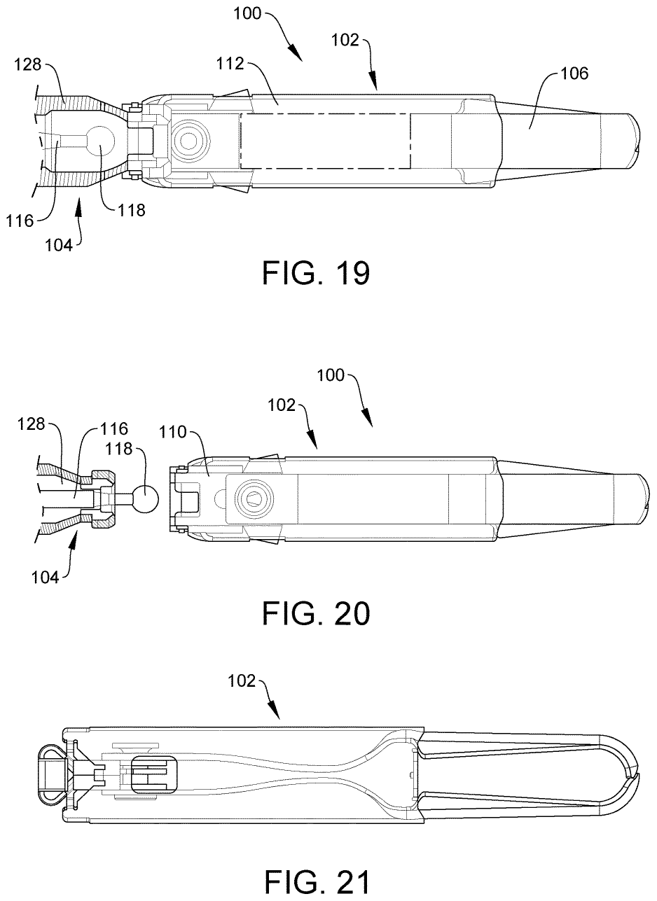

Thus, once the core member 110 is locked with respect to the capsule 112, to finally deploy the clip 102, the user may continue to draw the control member 116 proximally until the force exerted therealong exceeds the predetermined threshold value, causing the proximal opening 180 to deform, allowing the enlarged distal end 118 to pass therethrough, as shown in FIG. 19. As the tabs 136 of the capsule 112 are disengaged from the engaging portions 142 of the bushing 128, the enlarged distal end 118 of the control member 116 may be used to push the clip 102 distally away from the insertion device 104, separating the clip 102 from the bushing 128, as shown in FIG. 20. The insertion device 104 and the control member 116 may then be withdrawn from the body leaving just the clip 102, as shown in FIG. 21, clipped over the target tissue.

It will be apparent to those skilled in the art that various modifications may be made in the present disclosure, without departing from the scope of the disclosure. Although specific embodiments have been illustrated and described herein, it should be appreciated that any arrangement calculated to achieve the same purpose may be substituted for the specific embodiments shown. This disclosure is intended to cover any and all adaptations or variations of various embodiments. It is to be understood that the above description has been made in an illustrative fashion, and not a restrictive one. Combinations of the above embodiments, and other embodiments not specifically described herein will be apparent to those of skill in the art upon reviewing the above description. Thus, the scope of various embodiments includes any other applications in which the above compositions, structures, and methods are used.

What is claimed is:

1. A device for clipping tissue, comprising:
a capsule extending longitudinally from a proximal end to a distal end and including a channel extending therethrough;
a pair of clip arms, proximal ends of which are slidably received within the channel of the capsule to move the clip arms between an open configuration, in which distal ends thereof are separated from one another, and a closed configuration, in which the distal ends thereof are drawn toward one another; and
a core member connecting the proximal ends of the clip arms and slidably received within the capsule to move the clip arms between the open and closed configurations, the core member formed of a stamped sheet of metal that is bent into a configuration including a proximal portion defining a cavity configured to receive an enlarged distal end of a control member therewithin and a distal portion including first and second planar surfaces between which a pair of locking wings are received, the proximal portion includes a proximal opening through which a remaining length of the control member is configured to extend, the proximal opening configured to deform to permit a proximal passage of the enlarged distal end therethrough during a deployment of the device when subject to a force exceeding a predetermined threshold value, each of the locking wings connected to at least one of the first and second planar surfaces via a bent portion which deforms during movement of the locking wings from an unlocked configuration toward a locked configuration in which the locking wings are moved radially outward to engage a corresponding locking structure of the capsule, a length of the bent portion selected so that a force required to move the locking wings from the unlocked configuration toward the locked configuration is less than the predetermined threshold value for deforming the proximal opening.

2. The device of claim 1, wherein the proximal end of the capsule includes a pair of tabs crimped inward, toward a centerline of the capsule.

3. The device of claim 2, wherein the proximal portion of the core member includes a first flap and a second flap defining a portion of the cavity, the first and second flaps angled with respect to a longitudinal axis of the core member, a distance between distal ends of the first and second flaps configured so that, when the first and second flaps slide into contact with the tabs of the capsule, the tabs are pushed radially outward.

4. The device of claim 3, wherein the locking structure of the capsule includes a pair of locking windows extending through a wall thereof, each of the locking windows tapering toward a proximal end thereof to ensure that, when the locking wings are received within the locking windows, the first and second flaps are aligned with the tabs of the capsule to push the tabs radially outward.

5. The device of any one of claims 1-4, wherein the clip arms are connected to the core member via a rivet received within holes extending through the proximal end of the clip arms and through elongate openings extending through the first and second planar surfaces of the distal portion of the core member.

6. The device of claim 5, wherein, in the unlocked configuration, a portion of the locking wings overlap a distal portion of the elongate openings.

7. The device of claim 6, wherein the rivet is slidable from a proximal end of the elongate openings toward a distal end to move the locking wings from the unlocked configuration toward the locked configuration.

8. The device of claim 5, wherein interior longitudinal edges of the locking wings extend along one of a curve and an angle relative to a longitudinal axis of the core member selected so that, when the rivet is slid distally therealong, the locking wings are forced toward the locking configuration.

9. A system for clipping tissue, comprising:
an insertion device including a catheter extending longitudinally from a proximal end to a distal end and a bushing configured to be mounted over the distal end of the catheter;
a clip including a capsule releasably coupled to the catheter via the bushing and a pair of clip arms, proximal ends of which are connected to one another via a core member slidably received within a channel of the capsule to move the clip arms between an open configuration, in which distal ends of the clip arms are separated from one another to receive a tissue therebetween, and a closed configuration, in which distal ends of the clip arms are drawn toward one another to grip a tissue therebetween, the core member formed of a stamped sheet of metal that is bent into a configuration including a proximal portion defining a cavity and a distal portion including first and second planar surfaces between which a pair of locking wings are received, each of the locking wings connected to at least one of the first and second planar surfaces via a bent portion configured to deform when the locking wings is moved from an unlocked configuration toward a locked configuration, in which the locking wings are moved radially outward to engage a corresponding locking structure of the capsule; and
a control member including an enlarged distal end received within the cavity of the core member and a remaining length passing through a proximal opening of the core member to extend through the bushing and catheter to a proximal end of the insertion device, the proximal opening configured to deform to permit a proximal passage of the enlarged distal end therethrough during a deployment of the clip, when subject to a force exceeding a predetermined threshold value, a length of the bent portion selected so that a force required to move the locking wings from the unlocked configuration toward the locked configuration is less than the predetermined threshold value for deforming the proximal opening.

10. The system of claim 9, wherein the bushing includes a dimple projecting into a channel of the bushing, the dimple sized, shaped, and configured to prevent the distal end of the catheter from moving distally therepast.

11. The system of any one of claims 9-10, wherein the capsule includes a pair of tabs crimped radially inward configured to engage corresponding engaging feature at a distal end of the bushing.

12. The system of claim 11, wherein the proximal portion of the core member includes a first and second flap configured to engage the tabs of the capsule to move the tabs radially outward.

13. The system of claim 12, the capsule including a pair of locking windows extending through a wall of the capsule and configured to receive the locking wings therein, in the locked configuration, each of the locking windows tapering toward a proximal end thereof so that, when the locking wings engage the locking windows, the core member is accurately aligned with the capsule to ensure that the first and second tabs engage the tabs to move the tabs radially outward.

14. The system of any one of claims 9-13, wherein the clip further comprises a rivet received within holes extending through proximal ends of the clip arms and through elongated openings extending through the first and second planar surfaces to connect the clip arms to the core member.

15. The system of claim 14, wherein the rivet is movable from a proximal end of the elongated openings toward a distal end of the elongated openings to engage a portion of the locking wings to slide along a portion of the locking wings overlapping a distal portion of the elongated openings to push the locking wings radially outward toward the locking configuration.

16. A device for clipping tissue, comprising:
a capsule extending longitudinally from a proximal end to a distal end and including a channel extending therethrough;
a pair of clip arms, proximal ends of which are slidably received within the channel of the capsule to move the clip arms between an open configuration, in which distal ends thereof are separated from one another, and a closed configuration, in which the distal ends thereof are drawn toward one another; and
a core member connecting the proximal ends of the clip arms and slidably received within the capsule to move the clip arms between the open and closed configurations, the core member formed of a stamped sheet of metal that is bent into a configuration including a proximal portion defining a cavity configured to receive an enlarged distal end of a control member therewithin and a distal portion including first and second planar surfaces between which a pair of locking wings are received, the proximal portion includes a proximal opening through which a remaining length of the control member is configured to extend, the proximal opening configured to deform to permit a proximal passage of the enlarged distal end therethrough during a deployment of the device when subject to a force exceeding a predetermined threshold value, each of the locking wings connected to at least one of the first and second planar surfaces via a bent portion which deforms during movement of the locking wings from an unlocked configuration toward a locked configuration in which the locking wings are moved radially outward to engage a corresponding locking structure of the capsule, a length of the bent portion selected so that a force required to move the locking wings from the unlocked configuration toward the locked configuration is less than the predetermined threshold value for deforming the proximal opening.

17. The device of claim 16, wherein the proximal end of the capsule includes a pair of tabs crimped inward, toward a centerline of the capsule.

18. The device of claim 17, wherein the proximal portion of the core member includes a first flap and a second flap defining a portion of the cavity, the first and second flaps angled with respect to a longitudinal axis of the core member, a distance between distal ends of the first and second flaps configured so that, when the first and second flaps slide into contact with the tabs of the capsule, the tabs are pushed radially outward.

19. The device of claim 18, wherein the locking structure of the capsule includes a pair of locking windows extending through a wall thereof, each of the locking windows tapering toward a proximal end thereof to ensure that, when the locking wings are received within the locking windows, the first and second flaps are aligned with the tabs of the capsule to push the tabs radially outward.

20. The device of claim 16, wherein the clip arms are connected to the core member via a rivet received within holes extending through the proximal end of the clip arms and through elongate openings extending through the first and second planar surfaces of the distal portion of the core member.

21. The device of claim 20, wherein, in the unlocked configuration, a portion of the locking wings overlap a distal portion of the elongate openings.

22. The device of claim 21, wherein the rivet is slidable from a proximal end of the elongate openings toward a distal end to move the locking wings from the unlocked configuration toward the locked configuration.

23. The device of claim 20, wherein interior longitudinal edges of the locking wings extend along one of a curve and an angle relative to a longitudinal axis of the core member selected so that, when the rivet is slid distally therealong, the locking wings are forced toward the locking configuration.

24. A system for clipping tissue, comprising:
an insertion device including a catheter extending longitudinally from a proximal end to a distal end and a bushing configured to be mounted over the distal end of the catheter;
a clip including a capsule releasably coupled to the catheter via the bushing and a pair of clip arms, proximal ends of which are connected to one another via a core member slidably received within a channel of the capsule to move the clip arms between an open configuration, in which distal ends of the clip arms are separated from one another to receive a tissue therebetween, and a closed configuration, in which distal ends of the clip arms are drawn toward one another to grip a tissue therebetween, the core member formed of a stamped sheet of metal that is bent into a configuration including a proximal portion defining a cavity and a distal portion including first and second planar surfaces between which a pair of locking wings are received, each of the locking wings connected to at least one of the first and second planar surfaces via a bent portion configured to deform when the locking wings is moved from an unlocked configuration toward a locked configuration, in which the locking wings are moved radially outward to engage a corresponding locking structure of the capsule; and
a control member including an enlarged distal end received within the cavity of the core member and a remaining length passing through a proximal opening of the core member to extend through the bushing and catheter to a proximal end of the insertion device, the proximal opening configured to deform to permit a proximal passage of the enlarged distal end therethrough during a deployment of the clip, when subject to a force exceeding a predetermined threshold value, a length of the bent portion selected so that a force required to move the locking wings from the unlocked configuration toward the locked configuration is less than the predetermined threshold value for deforming the proximal opening.

25. The system of claim 24, wherein the bushing includes a dimple projecting into a channel of the bushing, the dimple sized, shaped, and configured to prevent the distal end of the catheter from moving distally therepast.

26. The system of claim 24, wherein the capsule includes a pair of tabs crimped radially inward configured to engage corresponding engaging feature at a distal end of the bushing.

27. The system of claim 26, wherein the proximal portion of the core member includes a first and second flap configured to engage the tabs of the capsule to move the tabs radially outward.

28. The system of claim 27, the capsule including a pair of locking windows extending through a wall of the capsule and configured to receive the locking wings therein, in the locked configuration, each of the locking windows tapering toward a proximal end thereof so that, when the locking wings engage the locking windows, the core member is accurately aligned with the capsule to ensure that the first and second tabs engage the tabs to move the tabs radially outward.

29. The system of claim 24, wherein the clip further comprises a rivet received within holes extending through proximal ends of the clip arms and through elongated openings extending through the first and second planar surfaces to connect the clip arms to the core member.

30. The system of claim 29, wherein the rivet is movable from a proximal end of the elongated openings toward a distal end of the elongated openings to engage a portion of the locking wings to slide along a portion of the locking wings overlapping a distal portion of the elongated openings to push the locking wings radially outward toward the locking configuration.

31. A method for treating target tissue, comprising:
inserting a clip through a working channel of an endoscope to a target site within a body via a catheter releasably coupled to the clip via a bushing, the clip including a capsule and clip arms, proximal end of the clip arms connected to one another via a core member slidably received within the capsule;
moving the clip between an open configuration, in which distal ends of the clip arms are separated from one another, to a closed configuration, in which the distal ends of the clip arms are drawn toward one another, until a target tissue is clipped between the clip arms, as desired, the clip moved between the open and closed configurations by moving a control member coupled to the core member distally and proximally relative to the capsule, an enlarged distal end of the control member received within a cavity of the core member and a remaining length of the control member extending proximally from the core member via a proximal opening of the core member coupled to the core member;
drawing the control member proximally relative to the capsule until engaging features along a distal portion of the clip arms abut a distal face of the capsule preventing further proximal movement of the clip arms relative to the capsule;
locking the clip arms toward the closed configuration, clipped over the target tissue, by drawing the control member further proximally relative to the capsule until a rivet received within openings at proximal ends of the clip arms and within an elongated opening of the core member is moved from a proximal end of the elongated opening toward a distal end of the elongated opening, sliding along interior longitudinal edges of a pair of locking wings to force the locking wings radially outward toward a locked configuration in which the locking wings extend into locking windows of the capsule; and
deploying the clip by drawing the control member further proximally relative to the capsule until a force exerted on a proximal opening of the control member via the enlarged distal exceeds a predetermined threshold value such that the proximal opening deforms to permit a proximal passage of the enlarged distal end of the control member therethrough.

32. The method of claim 31, wherein the bushing is mounted over the distal end of the catheter, the bushing including a dimple projecting into a channel thereof to prevent the distal end of the catheter from moving distally therepast.

33. The method of claim 31, wherein when the locking wings of the core member are received within the locking windows of the capsule, first and second flaps along a proximal portion of the core member engage tabs at a proximal end of the capsule, which are crimped radially inward to engage a corresponding portion of the bushing, to move the tabs radially outward, out of engagement with the corresponding portion of the bushing.

34. The method of claim 33, wherein the locking windows taper toward a proximal end thereof to ensure appropriate alignment of the first and second flaps of the core member with the tabs of the capsule.

35. The method of claim 31, wherein the core member is formed of a stamped sheet of metal that is bent into a configuration including a proximal portion defining the cavity and a distal portion connected to the proximal end of the clip arms, the distal portion including first and second planar surfaces between which the locking wings are received, each of the locking wings connected to at least one of the first and second planar surfaces via a bent portion, a length of the bent portion selected so that a force required to move the locking wings from an unlocked configuration toward the locked configuration is less than the predetermined threshold value required to deform the proximal opening of the core member during clip deployment.
